(19) United States
(12) Patent Application Publication
(10) Pub. No.: US 2021/0059740 A1
(43) Pub. Date: Mar. 4, 2021

(54) LOCALIZED CRYOTHERAPY SYSTEMS AND METHODS

(71) Applicant: IsoCryo, LLC, Auburn, AL (US)

(72) Inventors: John H. Mann, III, Auburn, AL (US); Johnny Mann, Auburn, AL (US)

(73) Assignee: IsoCryo, LLC, Auburn, AL (US)

(21) Appl. No.: 17/007,960

(22) Filed: Aug. 31, 2020

Related U.S. Application Data
(60) Provisional application No. 62/894,453, filed on Aug. 30, 2019.

(51) Int. Cl. A61B 18/02; A61B 90/00

(52) U.S. Cl. CPC A61B 18/0218; A61B 2018/00791; A61B 90/06

(57) ABSTRACT

Systems and methods for localized cryotherapy treatments with improved gas delivery and safety features are disclosed. The systems and methods incorporate a high-pressure supply of cryogenic fluid that is dispersed through an atomizing nozzle. Utilization of a high-pressure supply of cryogenic liquid allows the delivery of cryogenic fluid to a user's skin without the need to heat or otherwise increase the thermal energy of the cryogenic fluid prior to dispersion. The systems and methods also incorporate a thermographic imaging camera to measure the body surface temperature of a patient during the cryotherapy treatment. The thermographic imaging camera can measure the body surface temperature from a distance, which reduces the risk of inaccurate readings due to ambient temperature changes and improves the safety of the patient.

### CROSS REFERENCE TO RELATED APPLICATIONS

This application claims the benefit of and priority to U.S. Provisional Application No. 62/894,453, filed on Aug. 30, 2019, and entitled “Localized Cryotherapy Device Systems and Methods,” the disclosure of which is expressly incorporated by reference in its entirety.

### FIELD OF THE DISCLOSURE

The present disclosure relates generally to cryotherapy, and more particularly to systems and methods for localized cryotherapy with improved gas delivery.

### BACKGROUND

Localized cryotherapy is becoming more popular to treat a number of ailments ranging from weight loss to inflammation to muscle pain. Most localized cryotherapy systems utilize a cryotherapy machine comprised of a base and a handheld wand or applicator. The handheld wand typically contains a nozzle, trigger mechanism, and light emitting diode (LED) or laser light for aiming the device at the treatment area. The base typically contains a tank of low-pressure cryogenic liquid, a heater, and control electronics. The heater is often electronically powered and heats the cryogenic liquid so that the liquid is converted to a cryogenic gas.

One of the leading concerns for localized cryotherapy treatment is the risk of exposing the patient's skin to the cryogenic liquid. Heaters encourage the liquid to convert to a gas before the fluid is discharged. However, small amounts of cryogenic liquid sometimes still reach the handheld unit (and subsequently, the patient receiving the treatment). Such exposure can cause integumentary damage such as frost bite and burns that can be extremely harmful to the client.

Accordingly, there remains a need in the art for a localized cryotherapy system with improved gas delivery and safety features to reduce the risk of patient injury.

### SUMMARY

Systems and methods for localized cryotherapy treatments with improved gas delivery and safety features are disclosed. The systems and methods of the present disclosure utilize a high-pressure supply of cryogenic fluid through the use of an atomizing nozzle, which allows the delivery of cryogenic fluid to a user's skin without the need to heat or otherwise increase the thermal energy of the cryogenic fluid prior to dispersion. The high-pressure supply of cryogenic fluid also provides for shorter treatment times. The systems and methods further incorporate a thermographic imaging camera to measure the body surface temperature of a patient during the cryotherapy treatment. The thermographic imaging camera can measure the body surface temperature from a distance, which reduces the risk of inaccurate readings due to ambient temperature changes and improves the safety of the patient. Moreover, the systems and methods of the present disclosure allow for the cryogenic hose to be connected directly to the main storage tank, which dispenses of the need for any intermediary cryogenic fluid tanks.

In some embodiments, a localized cryotherapy system is provided, the localized cryotherapy system including a tank for storing cryogenic fluid; a cryogenic hose having a first end operatively connected to the tank and a second end operatively connected to a handheld unit, wherein the handheld unit includes an atomizing nozzle; a valve in fluid communication with the cryogenic hose and operatively connected to a control system; wherein the control system is operatively connected to one or both of a control input interface and a thermographic imaging camera, the control system configured to receive a signal from one or both of the control input interface and the thermographic imaging camera and communicate an instruction to the valve to adjust the flow of the cryogenic fluid. In one embodiment, the atomizing nozzle includes an orifice having a diameter of about 0.042 inches to about 0.076 inches. In another embodiment, the thermographic imaging camera is operatively connected to a control screen, the control screen configured to display body surface temperatures measured by the thermographic imaging camera. The thermographic imaging camera may further include a laser configured to pinpoint a location at which the body surface temperature is to be measured during the cryotherapy. In still another embodiment, the valve may be an electrically actuated solenoid valve, a motor actuated valve, or an electronic globe valve. In yet another embodiment, the signal includes body surface temperatures measured by the thermographic imaging camera, inputs from the control input interface, or combinations thereof. In another embodiment, the tank is configured to store the cryogenic fluid at a pressure of about 100 psi to about 500 psi.

In other embodiments, a localized cryotherapy system is provided, the localized cryotherapy system including a tank for storing cryogenic fluid at a pressure of at least about 100 psi; a first cryogenic hose having a first end operatively connected to the tank and a second end operatively connected to a valve; a second cryogenic hose having a first end operatively connected to the valve and a second end operatively connected to a handheld unit, wherein the handheld unit includes an atomizing nozzle; a first mobile station including a control system operatively connected to the valve; a second mobile station including a thermographic imaging camera configured for measuring body surface temperatures during cryotherapy, and wherein the control system is configured to receive the measured body surface temperatures from the thermographic imaging camera and communicate a signal to the valve to increase, decrease, or stop the flow of the cryogenic fluid. In one embodiment, the first mobile station further includes a control input interface operatively connected to the control system. In another embodiment, the second mobile station further includes a control screen operatively connected to the thermographic imaging camera, the control screen configured to display body surface temperatures measured by the thermographic imaging camera. The first mobile station and the second mobile station may each be battery powered. In still another embodiment, the valve includes an electrically actuated solenoid valve, a motor actuated valve, or an electronic globe valve. In yet another embodiment, the tank is configured to store the cryogenic fluid at a pressure of up to about 500 psi. In another embodiment, the handheld unit further includes a depth sensor configured to accurately position the handheld unit at an optimum distance from a user during the cryotherapy. In still another embodiment, the atomizing nozzle includes an orifice having a diameter of about 0.042 inches to about 0.076 inches.

In still other embodiments, a method for cryotherapy treatment is provided, the method including supplying a flow of cryogenic fluid from a tank to a handheld unit including an atomizing nozzle; dispersing the cryogenic fluid through the atomizing nozzle to ambient air to provide a cryotherapy treatment to a patient; measuring, with a thermographic imaging camera, the patient's body surface temperature during the cryotherapy treatment; and adjusting the flow of cryogenic fluid based on body surface temperature measurements obtained from the thermographic imaging camera. In one embodiment, the measuring step further includes displaying the patient's measured body surface temperature on a control screen. In another embodiment, the adjusting step further includes increasing, decreasing, or stopping the flow of cryogenic fluid based on the patient's measured body surface temperature. In still another embodiment, the measuring step further includes positioning the thermographic imaging camera at least about two to five feet away from the patient. In yet another embodiment, the supplying step further includes supplying the flow of cryogenic fluid from a tank configured to store the cryogenic fluid at a pressure of about 100 psi to about 500 psi.

### BRIEF DESCRIPTION OF THE DRAWINGS

Further features and advantages can be ascertained from the following detailed description that is provided in connection with the drawings described below:

FIG. 1A is a schematic diagram showing a localized cryotherapy system in accordance with an embodiment of the present disclosure.

FIG. 1B is a schematic diagram showing the localized cryotherapy system in accordance with another embodiment of the present disclosure.

FIG. 2 is a schematic diagram of a control system for controlling the flow of cryogenic fluid according to one embodiment of the present disclosure.

FIG. 3 is an interior view of a mobile cart utilized in the localized cryotherapy system of FIG. 1A according to one embodiment.

FIG. 4 is a perspective view of the mobile cart utilized in the localized cryotherapy system of FIG. 1A according to an embodiment of the present disclosure.

FIG. 5 is a perspective view of a handheld unit utilized in the localized cryotherapy system of FIG. 1A according to one embodiment.

FIG. 6 is a perspective view of an atomizing nozzle in accordance with an embodiment of the present disclosure.

FIG. 7 is a perspective view of an adapter for use with the atomizing nozzle of FIG. 6 according to one embodiment of the present disclosure.

FIG. 8A is a perspective view of a thermographic imaging camera and control screen according to one embodiment of the present disclosure.

FIG. 8B is a perspective view of the thermographic imaging camera according to another embodiment of the present disclosure.

FIG. 9 is a perspective view of the mobile cart utilized in the localized cryotherapy system of FIG. 1A and a second mobile cart equipped with the thermographic imaging camera and control screen of FIG. 8A according to one embodiment of the present disclosure.

FIG. 10 is a flowchart illustrating the steps according to a method for cryotherapy treatment in accordance with an embodiment of the present disclosure.

### DETAILED DESCRIPTION

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. Numerical quantities given in this description are approximate unless stated otherwise, meaning that the term “about” or “approximately” can be inferred when not expressly stated.

The terminology used herein is for the purpose of describing particular embodiments only and is not intended to be limiting. As used herein, the singular forms “a”, “an” and “the” are intended to include the plural forms as well (i.e., at least one of whatever the article modifies), unless the context clearly indicates otherwise.

Spatially relative terms, such as “under,” “below,” “lower,” “over,” “upper,” and the like, may be used herein for ease of description to describe one element or feature's relationship to another when the apparatus is right side up as shown in the accompanying drawings.

The terms “first,” “second,” “third,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The present disclosure relates generally to systems and methods for localized cryotherapy treatments with improved gas delivery and safety features. More particularly, the systems of the present disclosure produce a highly effective thermal energy transfer environment by utilizing a high-pressure supply of cryogenic fluid that is dispersed through an atomizing nozzle. Utilization of a high-pressure supply of cryogenic liquid allows the delivery of cryogenic fluid to a user's skin without the need to heat or otherwise increase the thermal energy of the cryogenic fluid prior to dispersion. This improved gas delivery system and method also promotes safer treatments, as it ensures that cryogenic liquid is rapidly and efficiently converted into cryogenic gas, thereby protecting users from harmful exposure to cryogenic liquid.

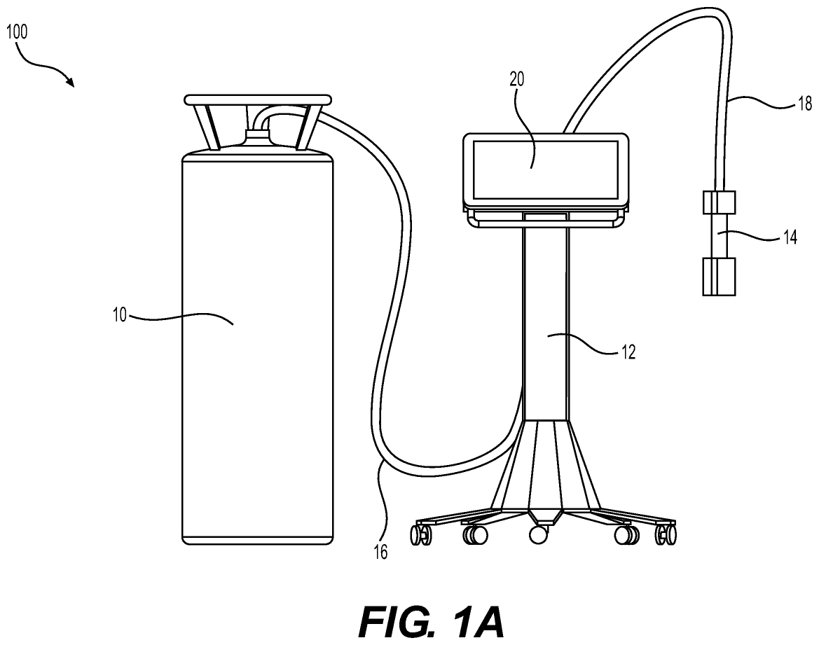

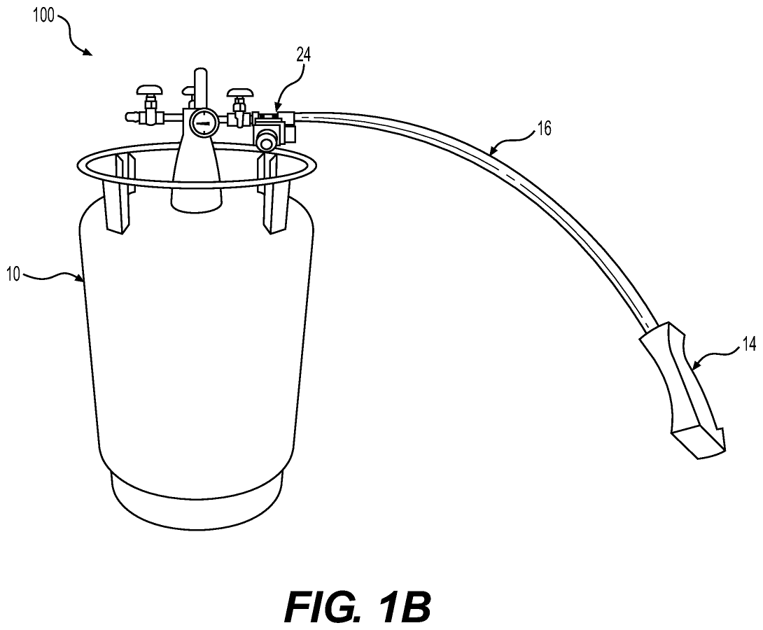

Referring to FIG. 1A, a localized cryotherapy system 100 according to an exemplary embodiment of the present disclosure is shown. The localized cryotherapy system 100 includes a storage tank 10 for storing a high-pressure supply of cryogenic fluid. As used herein, “cryogenic fluid” refers to any type of cold inert gas, such as liquid nitrogen or carbon dioxide. In some embodiments, the storage tank 10 may be a cryogenic storage dewar tank or a microbulk tank. The high-pressure supply of cryogenic fluid is generally stored in the storage tank 10 at a psi between about 100 psi and about 500 psi. In one embodiment, the high-pressure supply of cryogenic fluid is stored in the storage tank 10 at a psi of at least about 150 psi. In another embodiment, the high-pressure supply of cryogenic fluid is stored in the storage tank 10 at a psi of at least about 200 psi. In still another embodiment, the high-pressure supply of cryogenic fluid is stored in the storage tank 10 at a psi of about 180 psi to about 230 psi. In yet another embodiment, the high-pressure supply of cryogenic fluid is stored in the storage tank 10 at a psi of at least about 250 psi. Advantageously, the utilization of a high-pressure supply of cryogenic liquid allows the delivery of cryogenic fluid to a user's skin without the need to heat the cryogenic fluid prior to dispersion, which dispenses of the need for a heater in the system. In addition, the utilization of a high-pressure supply of cryogenic fluid provides shorter and more efficient treatment times.

While the cryogenic fluid has been described herein as a high-pressure supply, the localized cryotherapy system 100 may also utilize a low to medium pressure supply of cryogenic fluid. For instance, the cryogenic fluid may be stored in the storage tank 10 at a psi of about 22 psi to about 100 psi. In another embodiment, the cryogenic fluid may be stored in the storage tank 10 at a psi of about 50 psi to about 100 psi. In still another embodiment, the cryogenic fluid may be stored in the storage tank 10 at a psi of about 80 psi to about 100 psi.

The storage tank 10 is in fluid communication with a handheld unit 14. The handheld unit 14 includes one or more nozzles (not shown) for applying the cryogenic fluid to specific areas of the user's body. The storage tank 10 may be in fluid communication with the handheld unit 14 via one or more cryogenic hoses. In one embodiment, as shown in FIG. 1A, a first cryogenic hose 16 operatively connects the storage tank 10 to a mobile cart 12. The first cryogenic hose 16 enables the flow of high-pressure cryogenic fluid from the storage tank 10 to a supply valve (not shown) within the mobile cart 12. A second cryogenic hose 18 is operatively connected to the first cryogenic hose 16 at the supply valve within the mobile cart 12 and to the handheld unit 14. The second cryogenic hose 18 enables the flow of high-pressure cryogenic fluid from the supply valve to the handheld unit 14 for application of the cryogenic treatment.

The localized cryotherapy system 100 also includes a control input interface 20 operatively connected to a control system (not shown) for controlling the flow of cryogenic fluid from the storage tank 10 to the handheld unit 14. In the illustrated embodiment, the control input interface 20 is integrated into the mobile cart 12. In this embodiment, the control input interface 20 may be connected to and in communication with the control system through external hard wiring or other circuitry. However, in other embodiments, the control input interface 20 may be located entirely separate from the localized cryotherapy system 100. For example, the control input interface 20 may be integrated as an app on a smart phone or tablet such that the control input interface 20 is in wireless communication with the control system (for example, through a Wi-Fi connection).

In one embodiment, the control input interface 20 includes a touch screen display incorporating a graphical user interface. In another embodiment, the control input interface 20 may include a display screen, such as an electroluminescent (ELD) display, liquid crystal display (LCD), light emitting diode (LED) display (e.g., organic light emitting diode (OLED) or microLED), plasma display panel (PDP), or quantum dot (QLED) display, operatively connected to an external input device, such as a keyboard or touch pad. The flow of cryogenic fluid may be controlled directly through user inputs into the control input interface 20 or, alternatively, controlled by the control system based on pre-defined settings. The control input interface 20 can also be designed to provide information or options for other various features, such as timers, alarms, or visual alerts indicating fluid level in the storage tank 10.

FIG. 1B shows the localized cryotherapy system 100 according to another embodiment of the present disclosure. As shown in FIG. 1B, a single cryogenic hose may be used to operatively connect the storage tank 10 directly to the handheld unit 14. For instance, the first cryogenic hose 16 may extend from the storage tank 10 to the handheld unit 14. In this embodiment, the first cryogenic hose 16 enables the flow of high-pressure cryogenic fluid from the storage tank 10 to the handheld unit 14, such that no mobile cart is included. The supply valve 24 may be located at the storage tank 10, for instance, where the first cryogenic hose 16 is operatively connected to the storage tank 10, if no mobile cart is included.

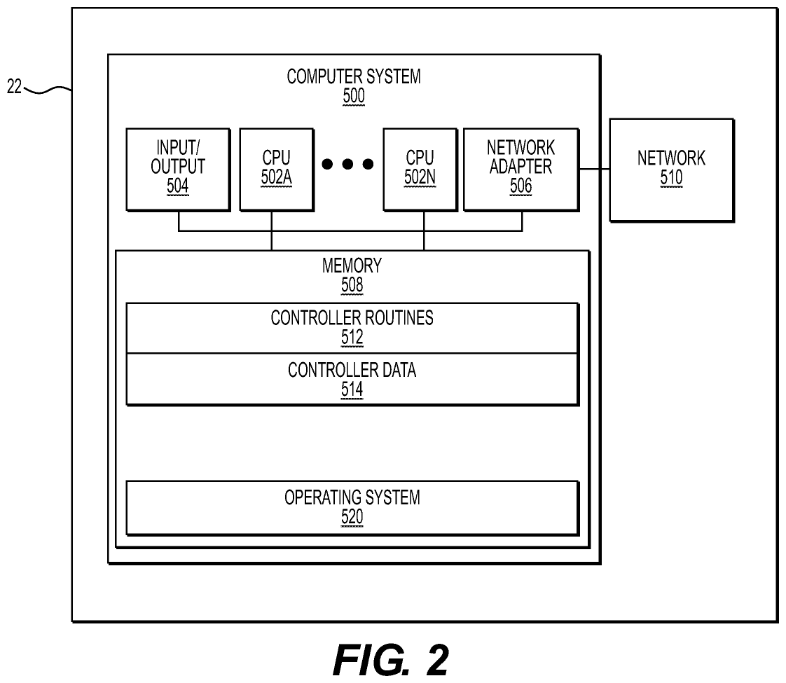

FIG. 2 is a schematic diagram of a control system 22 for controlling the flow of cryogenic fluid. The control system 22 may be integrated into the mobile cart 12. In this embodiment, as noted above, the control system 22 may be connected to and in communication with the control input interface 20 through hard wiring or wireless communication. In other embodiments, the control system 22 may be located external to the mobile cart 12. For example, the control system 22 may be located at any point along the first or second cryogenic hoses 16, 18 or near the storage tank 10 or the handheld unit 14.

Computer system 500 may typically be implemented using one or more programmed general-purpose computer systems, such as embedded processors, systems on a chip, personal computers, workstations, server systems, and minicomputers or mainframe computers, or in distributed, networked computing environments. Computer system 500 may include one or more processors (CPUs) 502A-502N, input/output circuitry 504, network adapter 506, and memory 508. CPUs 502A-502N execute program instructions to carry out the functions of the present systems and methods. Typically, CPUs 502A-502N are one or more microprocessors, such as an INTEL CORE® processor.

Input/output circuitry 504 provides the capability to input data to, or output data from, computer system 500. For example, input/output circuitry 504 may include input devices, such as the control input interface 20, keyboards, mice, touchpads, trackballs, scanners, and analog to digital converters; output devices, such as video adapters, monitors, and printers; and input/output devices, such as modems. Network adapter 506 interfaces computer system 500 with a network 510. Network 510 may be any public or proprietary data network, such as LAN and/or WAN (for example, the Internet).

Memory 508 stores program instructions that are executed by, and data that are used and processed by, CPU 502 to perform the functions of computer system 500. Memory 508 may include, for example, electronic memory devices, such as random-access memory (RAM), read-only memory (ROM), programmable read-only memory (PROM), electrically erasable programmable read-only memory (EEPROM), and flash memory, and electro-mechanical memory, which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, or Serial Advanced Technology Attachment (SATA), or a variation or enhancement thereof, or a fiber channel-arbitrated loop (FC-AL) interface.

Memory 508 may include controller routines 512, controller data 514, and operating system 520. Controller routines 512 may include software routines to perform processing to implement one or more controllers. Controller data 514 may include data needed by controller routines 512 to perform processing. In one embodiment, controller routines 512 may include software for analyzing and communicating incoming data from the control input interface 20 (for example, measurements related to the flow rate and the timing of the treatment). In another embodiment, controller routines 512 may include software for analyzing and communicating incoming data from a thermographic imaging camera (for example, measurements related to the surface temperature of the user's body), as will be discussed in more detail below. In still another embodiment, controller routines 512 may include software for analyzing and communicating incoming data from a depth sensor (for example, measurements related to the distance between the handheld unit 14 and the user), as will be described in more detail below.

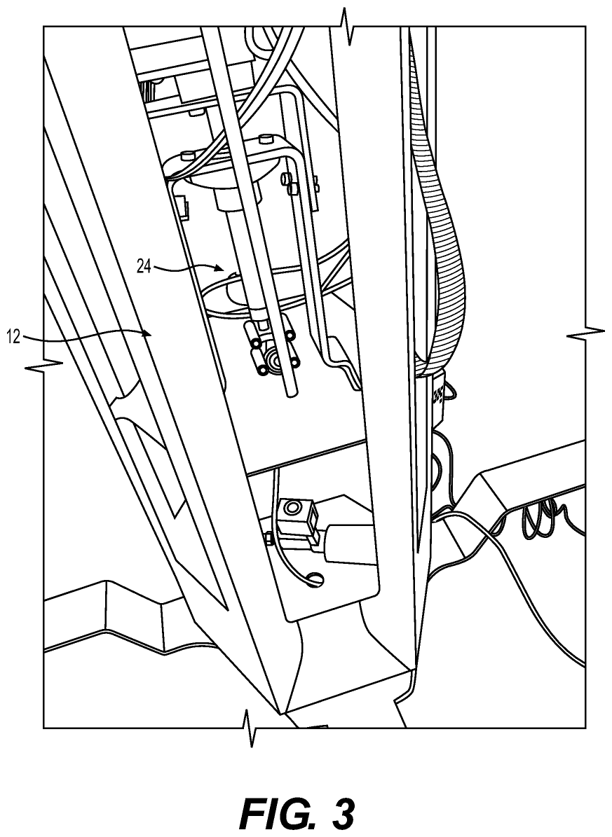

FIG. 3 shows an interior view of the mobile cart 12 of the localized cryotherapy system 100 illustrated in FIG. 1A. The control system 22 controls the flow of cryogenic fluid by a supply valve 24. Through user inputs into the control input interface 20 or communication with a thermographic imaging camera (as will be discussed in more detail below), the control system 22 can send signals to the supply valve 24 to increase, maintain, reduce, or stop the flow of cryogenic fluid. One or more supply valves 24 may be fluidly connected to the first and/or second cryogenic hose 16, 18. In one embodiment, the first cryogenic hose 16 and the second cryogenic hose 18 are adjustably connected within the mobile cart 12 by the supply valve 24. In another embodiment, the first cryogenic hose 16 is operatively connected to the storage tank 10 by the supply valve 24. When actuated by the control system 22, the supply valve 24 regulates the flow of cryogenic fluid from the storage tank 10 to the handheld unit 14. The supply valve 24 may be any type of valve or regulator configured to operate in response to control signals from the control system 22. For instance, as shown in FIG. 3, the supply valve 24 may be an electronic globe valve. In other embodiments, the supply valve 24 may be an electrically actuated solenoid valve (as shown in FIG. 1B) or a motor actuated valve. Exemplary supply valves 24 may include structures suitable for on/off valve operation and those that provide venting of the cryogenic fluid downstream of the valve when the flow is stopped to limit residual cryogenic fluid vaporization and cooling. In other embodiments, the supply valve 24 may be a manual valve that can be opened by pressing a handle or lever.

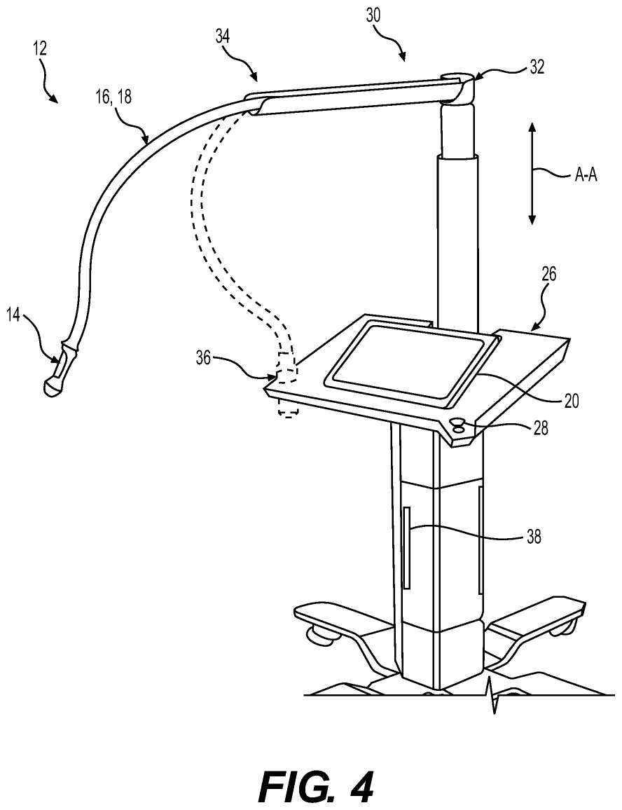

FIG. 4 is a perspective view of the mobile cart 12 according to an exemplary embodiment of the present disclosure. As illustrated in FIG. 4, the control input interface 20 is integrated on a surface 26 of the mobile cart 12, although the control input interface 20 may be positioned anywhere on the mobile cart 12 that is easily accessible by the user or other system operator during treatment. In one embodiment, the control input interface 20 may be adjustably attached to the surface 26 of the mobile cart 12. For instance, the control input interface 20 may be pivotally coupled to the surface 26 to allow for a user to adjust the control input interface 20 when in use. An emergency stop input 28 operably connected to the control system 22 may also be integrated in the surface 26 to cease the flow of cryogenic fluid.

In one embodiment, the mobile cart 12 may include a swing arm 30 for controlling the movement of the first or second cryogenic hose 16, 18 during use. As shown in FIG. 4, the swing arm 30 has a pivot end 32 operably connected to the mobile cart 12 and an opposite free end 34 for supporting the first or second cryogenic hose 16, 18. The swing arm 30 is movable or swingable towards and away from the mobile cart 12 via pivotal movement of the swing arm 30 about a generally vertical pivot axis A-A. The pivotal movement of the swing arm 30 allows for enhanced control by the user or system operator of the positioning of the first or second cryogenic hose 16, 18 during the cryotherapy treatment.

In another embodiment, the mobile cart 12 may further include a storage area 36 for storing the handheld unit 14 when not in use. As illustrated in FIG. 4, the storage area 36 may be located at an edge of the surface 26 such that the storage area 36 is easily accessible when the first or second cryogenic hose 16, 18 is connected to the swing arm 30. In other embodiments, the mobile cart 12 can include a panel access door 38 to allow a system operator access to the supply valve 24 and other aspects of the control system 22 that are situated within the mobile cart 12. Moreover, the mobile cart 12 may include visual design elements, such as LED lighting or glowing lights, to enhance the visual effects of the mobile cart 12 during the cryotherapy treatment. The mobile cart 12 may be battery-powered to provide increased mobility during treatment. In other embodiments, the mobile cart 12 can be powered by an external power source, such as by an electrical outlet.

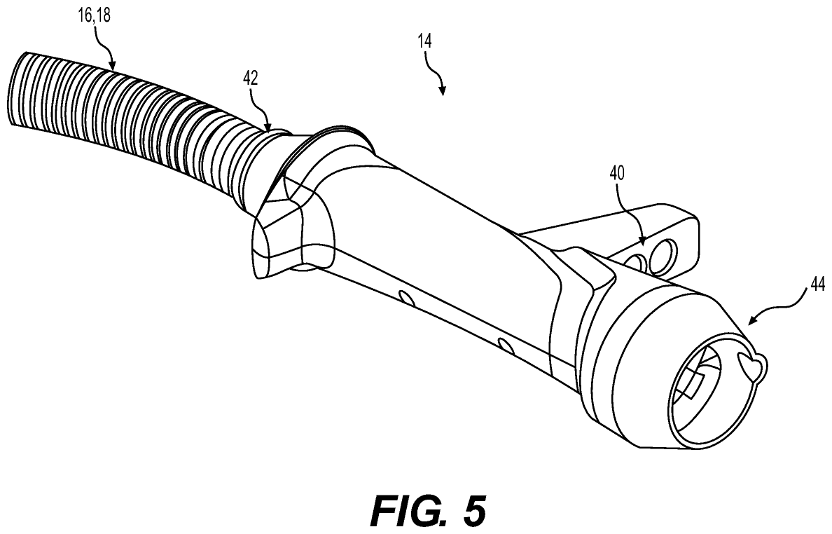

FIG. 5 illustrates the handheld unit 14 according to an exemplary embodiment of the present disclosure. The handheld unit 14 has a size and an ergonomic circular cross-sectional shape suitable for being grasped and supported in the hand of a user or other system operator. As shown in FIG. 5, the handheld unit 14 has an inlet 42 that is fluidly coupled to an end of the first or second cryogenic hose 16, 18 and an outlet 44 for discharging the cryogenic fluid. In one embodiment, the inlet 42 of the handheld unit 14 may have a threaded pipe that is configured for coupling to a corresponding threaded pipe or fitting on the end of the first or second cryogenic hose 16, 18. The outlet 44 may comprise a nozzle (not shown) that facilitates the discharge of cryogenic fluid for localized treatment. In one embodiment, the nozzle may include one or more atomizing nozzles, such as a hydraulic atomizing nozzle (as will be described in more detail below). In another embodiment, the nozzle may be any type of spray nozzle or mister nozzle. The handheld unit 14 may also include optical indicators (not shown) to aid in aiming the discharge of cryogenic fluid during treatment.

As illustrated in FIG. 5, the handheld unit 14 may also include a depth sensor 40. In this embodiment, the depth sensor 40 is configured to acquire distance information between the handheld unit 14 and the user's body part being treated by the cryotherapy. The depth sensor 40 may be used to accurately position the handheld unit 14 at the optimum distance from the user during treatment and prevent the handheld unit 14 from dispersing the cryogenic fluid too close to or too far away from the user's body. In one embodiment, the depth sensor 40 communicates with and transmits distance measurements to the control system 22. In this embodiment, the depth sensor 40 may include a light signal and/or an audible alert to communicate when the handheld unit 14 is out of an optimum distance range. The distance measurements and any resulting alerts may be displayed on the control input interface 20.

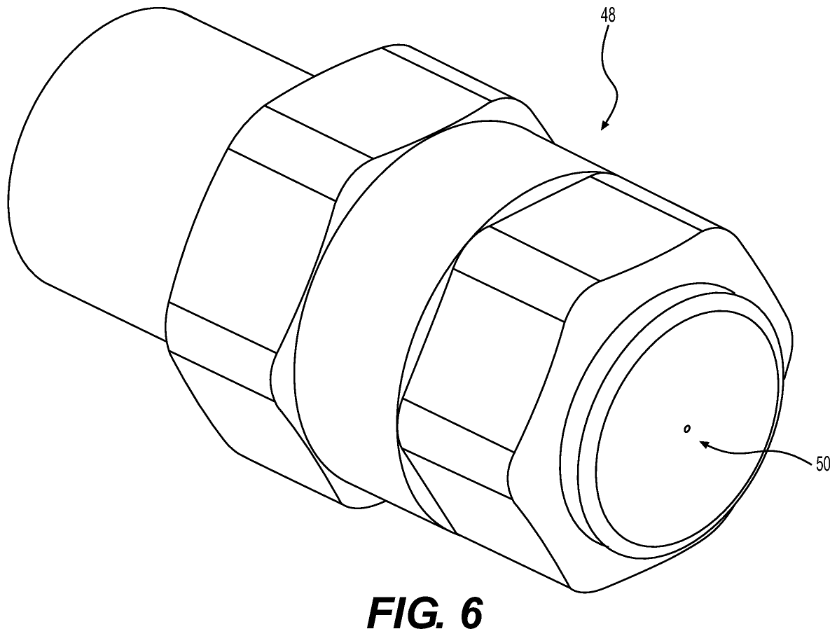

FIG. 6 is an atomizing nozzle 48 according to an exemplary embodiment of the present disclosure. The atomizing nozzle 48 may be fluidly coupled to the outlet 44 of the handheld unit 14 shown in FIG. 5. In another embodiment, the atomizing nozzle 48 may be fluidly coupled directly to an end of the first or second cryogenic hose 16, 18. Advantageously, atomization of the fluid using the atomizing nozzle 48 results in microscopic droplets that are evenly dispersed for uniform application and provides increased surface area to improve heat transfer from the ambient air. In the illustrated embodiment, the atomizing nozzle 48 has a single orifice 50 for discharging the cryogenic fluid. In other embodiments, the atomizing nozzle 48 may have a plurality of orifices designed to produce uniform droplets over an expanded target area.

The diameter of the orifice 50 may range from about 0.040 inches to about 0.080 inches. In another embodiment, the diameter of the orifice 50 may range from about 0.042 inches to about 0.076 inches. In still another embodiment, the diameter of the orifice 50 may range from about 0.050 inches to about 0.064 inches. Based on the range of diameters of the orifice 50 described herein, the atomizing nozzle 48 may have a flow rate capacity at 100 psi ranging from about 9.5 gallons per hour (0.16 gallons per minute) to about 19.0 gallons per hour (0.32 gallons per minute). In another embodiment, the atomizing nozzle 48 may have a flow rate capacity at 100 psi ranging from about 12.6 gallons per hour (0.21 gallons per minute) to about 15.8 gallons per hour (0.26 gallons per minute). Examples of suitable commercially available nozzles are nozzles of sizes 6-14 of the “LN” and the “N” models of the Fine Spray Hydraulic Atomizing Nozzles sold by Spraying Systems Co.®.

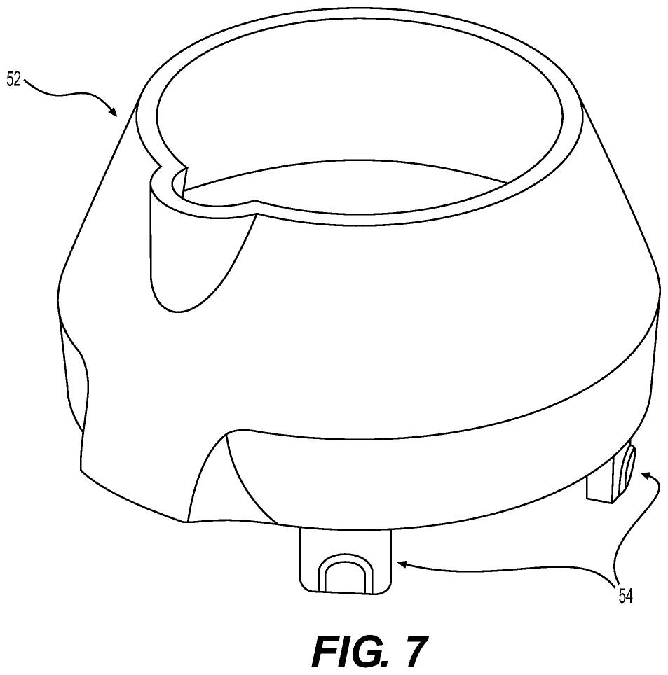

In some embodiments, the atomizing nozzle 48 may be fitted with various adapters to reduce or enlarge the surface area of the discharged droplets. FIG. 7 provides an example of an adapter 52 that may be coupled to the atomizing nozzle 48. The adapter 52 shown in FIG. 7 may be fitted to the atomizing nozzle 48 to enlarge the area and reduce the concentration of the dispersion. This adapter may be used, for instance, when applying the cryotherapy treatment to the face of the user or other sensitive body parts. In the illustrated embodiment, the adaptor 52 has a plurality of coupling members 54 for attachment to the atomizing nozzle 48 by a snap fit mechanism. However, the adaptor 52 may also be attached to the atomizing nozzle 48 by any other suitable securing means, such as by threaded coupling, screws, pins, projections, tongue and groove solutions, or snap catch elements.

The localized cryotherapy system 100 of the present disclosure may also incorporate a device for measuring and monitoring the surface temperature of the user's skin during treatment. In one embodiment, the localized cryotherapy system 100 incorporates a thermographic imaging camera to measure the temperature of the user's skin. In this embodiment, the thermographic imaging camera is positioned separate and apart from the handheld unit 14, for instance, on a second mobile cart. Measuring the surface temperature of a user during treatment at a distance away from the treatment area is preferred because measuring a user's skin temperature in close proximity to the handheld unit 14 during treatment can result in inaccurate readings due to frost, moisture, and emitting particulates causing changes in ambient temperature around the atomizing nozzle 48. Thus, separating the thermographic imaging camera from the handheld unit 14 and taking temperature readings at a distance reduces the risk of inaccurate readings due to ambient temperature changes and improves the safety of the user.

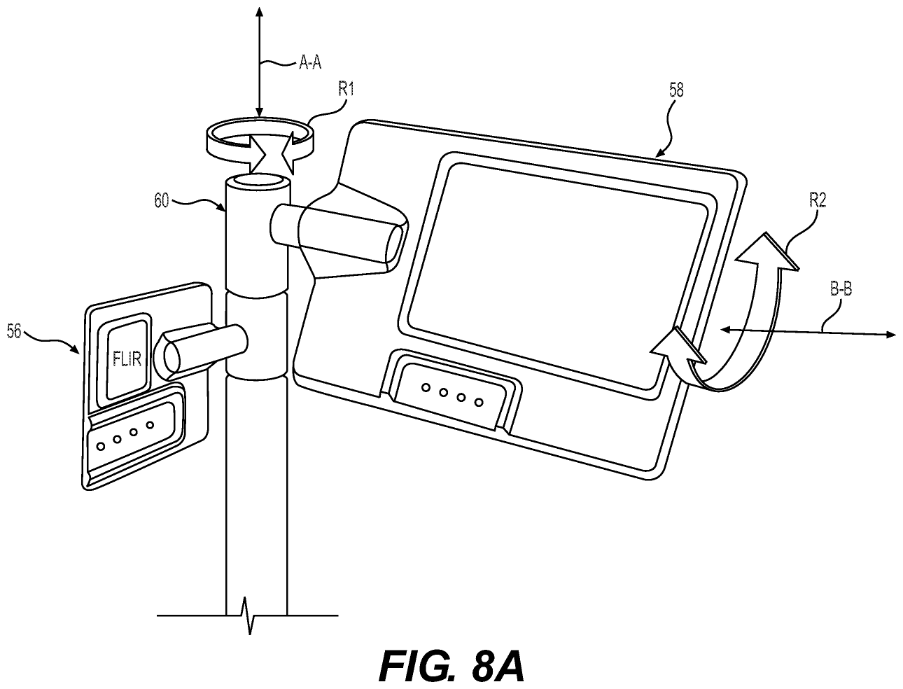

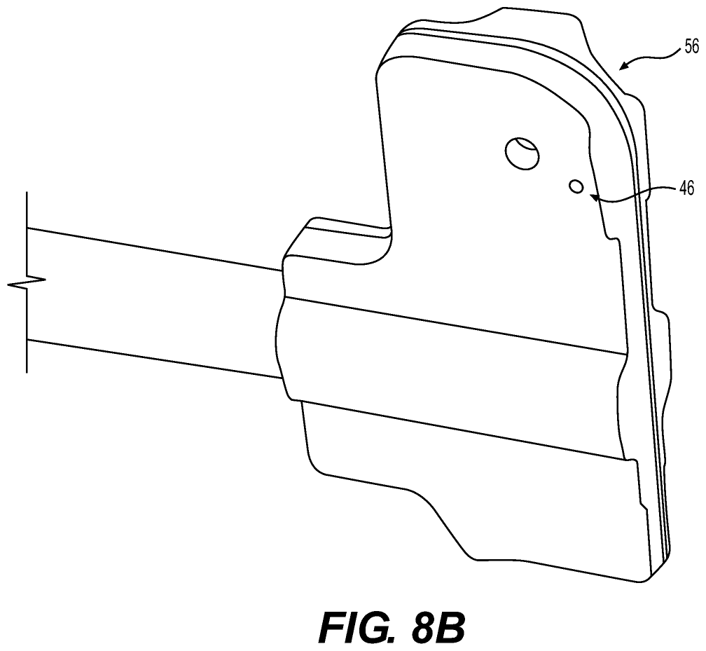

FIG. 8A shows a thermographic imaging camera 56 and a control screen 58 attached to a second mobile cart 60 in accordance with an exemplary embodiment of the present disclosure. The thermographic imaging camera 56 may be any type of thermal imaging infrared camera suitable for measuring the surface temperature of a human. The thermographic imaging camera 56 continuously monitors the surface temperature of the portion of the user's body undergoing treatment and transmits temperature measurements to the control system 22 such that the control system 22 may then account for the temperature readings to adjust the flow of cryogenic fluid during treatment. The thermographic imaging camera 56 remains in communication with the control system 22 to allow the control system 22 and/or the system operator (through the use of the control input interface 20) to adjust, increase, reduce, or stop the flow of cryogenic fluid as necessary to ensure client safety and to decrease or eliminate the risk of overexposure. The second mobile cart 60 may further include a shutoff control (not shown) in close proximity to the thermographic imaging camera 56 to stop the flow of cryogenic fluid when the temperature of the user's skin is outside of acceptable ranges. In one embodiment, the thermographic imaging camera 56 may communicate wirelessly with the control system 22, such as over a Wi-Fi network. In another embodiment, the thermographic imaging camera 56 may be connected to and in communication with the control system 22 through external hard wiring or other circuitry.

The control screen 58 may be any type of display screen, such as an ELD, LCD, LED, PDP, or QLED display or a touch screen display. In one embodiment, the control screen 58 is configured for displaying the user's surface temperature (for example, skin temperature). The control screen 58 can display thermal images of the user taken by the thermographic imaging camera 56. The thermal images may display body surface temperature by different color ranges. While the control screen 58 has been illustrated herein as a separate control screen, it is to be understood that the thermographic imaging camera 56 can be programmed such that the output data and graphics produced by the thermographic imaging camera 56 or otherwise developed using its measurements are also displayed on the control input interface 20.

The control screen 58 may be rotatably attached to the second mobile cart 60 such that the control screen 58 may be moved and manipulated by the system operator to allow both the system operator and the user to view the screen. For instance, as shown in FIG. 8A, the control screen 58 can rotate about vertical axis A-A (as represented by arrow R1). The user or system operator can adjust the control screen 58 by rotating the control screen 58 forward or backward. The control screen 58 can also rotate about horizontal axis B-B (as represented by arrow R2). In this embodiment, the user can adjust the control screen 58 by tilting the control screen 58 upward or downward. In one embodiment, the control screen 58 can be rotated about the horizontal axis B-B in a complete circle (i.e., 360 degrees).

FIG. 8B shows the thermographic imaging camera 56 according to another embodiment of the present disclosure. In the illustrated embodiment, the thermographic imaging camera 56 may incorporate an optical source 46, such as a laser, to produce one or more optical beams that illuminate a point on the user's body. The optical source 46 may be used to accurately position the thermographic imaging camera 56 at the optimum distance from the user. The optical source 46 may also be used to pinpoint a location on the user at which the temperature is to be measured during treatment. In other embodiments, the optical source 46 can illuminate the boundary or total area covered by the atomizing nozzle 48 so that the size of the area to be treated can be verified before applying treatment.

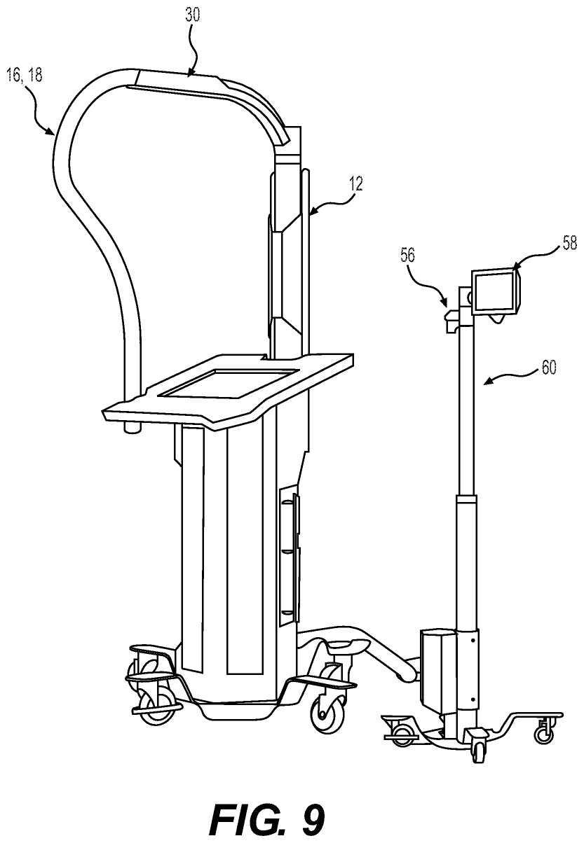

FIG. 9 shows an alternative embodiment of the present disclosure where the localized cryotherapy system 100 includes the mobile cart 12 and the second mobile cart 60 including the thermographic imaging camera 56 and the control screen 58. As shown in FIG. 9, the thermographic imaging camera 56 and the control screen 58 are mounted on the second mobile cart 60 so that they may be easily moved to an advantageous position for temperature monitoring depending on the body part of the user to be treated. Like the mobile cart 12, the second mobile cart 60 may be battery powered for increased mobility, although an external power source may also be used. The second mobile cart 60 allows for the thermographic imaging camera 56 to be positioned a sufficient distance away from the treatment area while measuring temperature. In one embodiment, the thermographic imaging camera 56 may be positioned at least about one to three feet away from the treatment area. In another embodiment, the thermographic imaging camera 56 may be positioned at least about two to five feet away from the treatment area. In still another embodiment, the thermographic imaging camera 56 may be positioned at least about four to six feet away from the treatment area, although measurements at distances greater than six feet may still be taken and used in accordance with the present disclosure. While the thermographic imaging camera 56 has been illustrated herein as attached to the second mobile cart 60, it is to be understood that the thermographic imaging camera 56 need not be attached to a separate mobile cart. The thermographic imaging camera 56 may be attached to the mobile cart 12 or positioned at a separate, fixed location altogether and still be incorporated into the localized cryotherapy system 100.

The various components of the localized cryotherapy system 100 described herein may be constructed or manufactured from materials, such as various polymers, plastics, stainless steel, aluminum, copper piping, brass piping, and combinations thereof. Similarly, the various parts described herein may be constructed according to various manufacturing methods including injection molding, milling, forging, extrusion, pressing, 3D printing, and other related manufacturing methods.

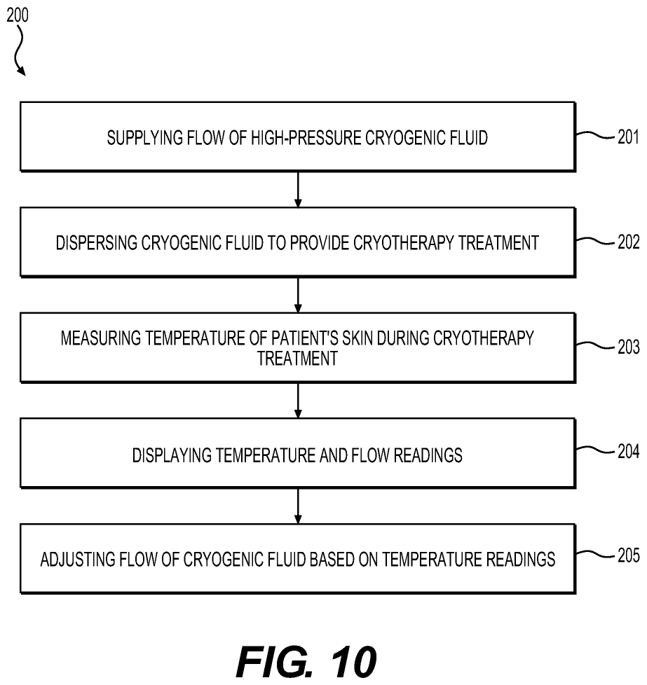

The present disclosure also provides methods for cryotherapy treatments incorporating the localized cryotherapy system 100 described herein. FIG. 10 illustrates one embodiment of a method for localized cryotherapy treatment 200 in accordance with the present disclosure. For example, step 201 includes supplying a flow of high-pressure cryogenic fluid from the storage tank 10 to the handheld unit 14. The high-pressure cryogenic fluid may be supplied through the use of the first cryogenic hose 16 and/or the second cryogenic hose 18. This step may further include controlling the flow of the high-pressure cryogenic fluid through the use of the control system 22. Step 202 includes dispersing the cryogenic fluid from the handheld unit 14 to ambient air to provide a cryotherapy treatment to a patient. In step 202, the dispersing step can further include atomizing the cryogenic fluid with one or more atomizing nozzles 48 provided on the handheld unit 14. Step 203 includes measuring the temperature of the patient's skin during the cryotherapy treatment. The measuring step can be performed by the thermographic imaging camera 56 and the resulting temperature measurements can be communicated to the control system 22. At step 204, information relating to the temperature readings and the flow of cryogenic fluid can be displayed to the patient or system operator on the control input interface 20 and/or the control screen 58. At step 205, the control system 22 and/or system operator can then increase, reduce, maintain, or stop the flow of cryogenic fluid based on the temperature readings.

The systems and methods described and claimed herein are not to be limited in scope by the specific embodiments herein disclosed, since these embodiments are intended as illustrations of several aspects of the disclosure. Any equivalent embodiments are intended to be within the scope of this disclosure. Indeed, various modifications of the systems and methods in addition to those shown and described herein will become apparent to those skilled in the art from the foregoing description. Such modifications are also intended to fall within the scope of the appended claims. All patents and patent applications cited in the foregoing text are expressly incorporated herein by reference in their entirety. Any section headings herein are provided only for consistency with the suggestions of 37 C.F.R. § 1.77 or otherwise to provide organizational queues. These headings shall not limit or characterize the invention(s) set forth herein.

What is claimed is:

1. A localized cryotherapy system, comprising:
a tank for storing cryogenic fluid;
a cryogenic hose having a first end operatively connected to the tank and a second end operatively connected to a handheld unit, wherein the handheld unit comprises an atomizing nozzle;
a valve in fluid communication with the cryogenic hose and operatively connected to a control system;
wherein the control system is operatively connected to one or both of a control input interface and a thermographic imaging camera, the control system configured to receive a signal from one or both of the control input interface and the thermographic imaging camera and communicate an instruction to the valve to adjust the flow of the cryogenic fluid.

2. The localized cryotherapy system of claim 1, wherein the atomizing nozzle comprises an orifice having a diameter of about 0.042 inches to about 0.076 inches.

3. The localized cryotherapy system of claim 1, wherein the thermographic imaging camera is operatively connected to a control screen, the control screen configured to display body surface temperatures measured by the thermographic imaging camera.

4. The localized cryotherapy system of claim 1, wherein the valve comprises an electrically actuated solenoid valve, a motor actuated valve, or an electronic globe valve.

5. The localized cryotherapy system of claim 1, wherein the signal comprises body surface temperatures measured by the thermographic imaging camera, inputs from the control input interface, or combinations thereof.

6. The localized cryotherapy system of claim 1, wherein the tank is configured to store the cryogenic fluid at a pressure of about 100 psi to about 500 psi.

7. The localized cryotherapy system of claim 1, wherein the thermographic imaging camera further comprises a laser configured to pinpoint a location at which the body surface temperature is to be measured during the cryotherapy.

8. A localized cryotherapy system, comprising:
a tank for storing cryogenic fluid at a pressure of at least about 100 psi;
a first cryogenic hose having a first end operatively connected to the tank and a second end operatively connected to a valve;
a second cryogenic hose having a first end operatively connected to the valve and a second end operatively connected to a handheld unit, wherein the handheld unit comprises an atomizing nozzle;
a first mobile station comprising a control system operatively connected to the valve;
a second mobile station comprising a thermographic imaging camera configured for measuring body surface temperatures during cryotherapy, and
wherein the control system is configured to receive the measured body surface temperatures from the thermographic imaging camera and communicate a signal to the valve to increase, decrease, or stop the flow of the cryogenic fluid.

9. The localized cryotherapy system of claim 8, wherein the first mobile station further comprises a control input interface operatively connected to the control system.

10. The localized cryotherapy system of claim 8, wherein the second mobile station further comprises a control screen operatively connected to the thermographic imaging camera, the control screen configured to display body surface temperatures measured by the thermographic imaging camera.

11. The localized cryotherapy system of claim 8, wherein the valve comprises an electrically actuated solenoid valve, a motor actuated valve, or an electronic globe valve.

12. The localized cryotherapy system of claim 8, wherein the tank is configured to store the cryogenic fluid at a pressure of up to about 500 psi.

13. The localized cryotherapy system of claim 8, wherein the handheld unit further comprises a depth sensor configured to accurately position the handheld unit at an optimum distance from a user during the cryotherapy.

14. The localized cryotherapy system of claim 8, wherein the first mobile station and the second mobile station are battery powered.

15. The localized cryotherapy system of claim 8, wherein the atomizing nozzle comprises an orifice having a diameter of about 0.042 inches to about 0.076 inches.

16. A method for cryotherapy treatment, comprising:
supplying a flow of cryogenic fluid from a tank to a handheld unit comprising an atomizing nozzle;
dispersing the cryogenic fluid through the atomizing nozzle to ambient air to provide a cryotherapy treatment to a patient;
measuring, with a thermographic imaging camera, the patient's body surface temperature during the cryotherapy treatment; and
adjusting the flow of cryogenic fluid based on body surface temperature measurements obtained from the thermographic imaging camera.

17. The method of claim 16, wherein the measuring step further comprises displaying the patient's measured body surface temperature on a control screen.

18. The method of claim 16, wherein the adjusting step further comprises increasing, decreasing, or stopping the flow of cryogenic fluid based on the patient's measured body surface temperature.

19. The method of claim 16, wherein the measuring step further comprises positioning the thermographic imaging camera at least about two to five feet away from the patient.

20. The method of claim 16, wherein the supplying step further comprises supplying the flow of cryogenic fluid from a tank configured to store the cryogenic fluid at a pressure of about 100 psi to about 500 psi.
